(19) United States
(12) Patent Application Publication
(10) Pub. No.: US 2022/0367015 A1
(43) Pub. Date: Nov. 17, 2022

(54) Biometric-Medical Emergency Data System, Med+ Card, and 911 Mobile Application

(71) Applicant: Carmen PICCINI, Santa Monica, CA (US)

(72) Inventor: Carmen PICCINI, Santa Monica, CA (US)

(21) Appl. No.: 17/556,724

(22) Filed: Dec. 20, 2021

Related U.S. Application Data
(63) Continuation of application No. 16/395,682, filed on Apr. 26, 2019, now abandoned.
Application No. 16/395,682 is a continuation of application No. 15/366,404, filed on Dec. 1, 2016, now abandoned.
Application No. 15/366,404 is a continuation-in-part of application No. 15/168,029, filed on May 28, 2016, now abandoned.
Application No. 15/168,029 is a continuation-in-part of application No. 14/995,226, filed on Jan. 14, 2016, now abandoned.

(51) Int. Cl. G16H 10/60; G06F 16/22; G06F 16/28; G06F 21/32; G06F 21/62

(52) U.S. Cl. CPC G16H 10/60; G06F 16/22; G06F 16/285; G06F 21/32; G06F 21/6245

(57) ABSTRACT

A method and system of collecting, organizing, and accessing medical data and records via a biometric read and scan. The system involves the implementation of a mechanism by which an individual's biometric signature is linked to his or her medical data, which is stored digitally and is accessible worldwide via a could-based database. The provided system is configured to be used in real-time, especially during emergency situations, to assist an individual who is not responding, and is in critical condition. The system includes multiple components, including a biometric reader/scanner device, a secured relational database which provides semantically categorized medical emergency content, a mobile device application, and applicable data encryption and security protocols. The mobile device application serves as a conduit for the user to contact the services of 911 instantly, as well as provide a service to the medical records of the user. The system includes a medical card.

### CONTINUITY

This is a Continuation patent application of non-provisional patent application Ser. No. 16/395,682, filed on Apr. 26, 2019 (the entire content of which is incorporated herein by reference in its entirety), which is a Continuation patent application of non-provisional patent application Ser. No. 15/366,404, filed on Dec. 1, 2016, which is a Continuation-in-Part of non-provisional patent application Ser. No. 15/168,029, filed on May 28, 2016, which is a Continuation-in-Part patent application of non-provisional patent application Ser. No. 14/995,226, filed on Jan. 14, 2016, and priority is claimed thereto.

### FIELD OF THE PRESENT INVENTION

The present invention relates to a system for logging biometric and medical data of individuals (including all women, men and children), and more specifically relates to a system for collecting, matching, storing, and providing universal access to the medical data of an individual via a biometric reading or scan of the individual, and rapidly conveying the data to all paramedics, first responders, all hospital doctors, authorized medical professionals, and medical personnel. More specifically, the present invention provides a system to be used in real-time—especially during EMERGENCY situations, to promptly assist any individual that is not responding and is in critical condition. In addition, the system of the present invention provides the option to include every individual's choice to donate organs—and the individual's specific DNR (Do Not Resuscitate) instructions and unique wishes of after-death instruction.

### BACKGROUND OF THE PRESENT INVENTION

After an emergency or catastrophe such as a tsunami, hurricane, tornado, flood, fire, or similar devastating event, medical professionals have difficulty identifying victims due in part to a lack of documentation on the victims. Additionally, bodily decay or destruction can make visual identification difficult. This is especially true in terrorist scenarios such as the events of 9/11, as well as earthquakes. This difficulty delays closure for families missing loved ones, and delays the recognition of organ donor status of the victims. Fingerprints are the last things to disappear when humans die. Even in the worst condition of decomposition, it is possible to verify fingerprints. In other circumstances, individuals may not have identification on their person, such as a driver's license or other government-issued ID card that can facilitate identification of the individual, leading to a delay in the retrieval of medical data by medical professionals. For example, if an individual drowns in the ocean, most likely he or she won't have an ID with him or herself; so, no one would know who the individual is.

Similarly, individuals that survive an emergency may be unable to convey medical restrictions and similar medical data to emergency response team members or other medical personnel, especially in cases where the individual is incapacitated or otherwise unconscious. This delays the retrieval of the medical history, records, and current medical status of the patient, often forcing a delay of treatment. As time is precious when it comes to medical services during an emergency, as we know the time-window is limited, and this delay can be troublesome. In some cases, when an individual is suffering an allergic reaction to treatment (which would have otherwise been known if the medical data of the patient was available), the application of the treatment without possession of the medical history of the patient can cause the death of the patient. If there were a way to rapidly retrieve the medical data of a patient from any point in the world with just a fingerprint of the individual, these delays would be minimized, facilitating immediate and accurate treatment of the patient when seconds can mean the difference between life and death. Therefore, biometric—and particularly in this case, biometric fingerprints—are very instrumental for identification.

U.S. Pat. No. 9,213,736, issued on Dec. 15, 2015, to Steves is for an “Operating System Fingerprinting.” Unlike the present invention, the invention taught by Steves contains different meanings and mechanisms as described in the process of the present invention.

U.S. Pat. No. 9,220,464, issued to Terai et al. on Dec. 29, 2015, is for a medical information management device. Unlike the present invention, Terai doesn't offer any similar improvement and process as described in the present invention. For example, Terai does not teach a method of employing biometric authentication in the field, such as at the scene of an emergency, to rapidly obtain medical data relating to the individual in need of medical care. Additionally, Terai does not teach the use of a supplemental Med+ Card to facilitate access to medical records in locations lacking cellular/internet wireless services.

U.S. Pat. No. 9,224,056 issued to Roemen on Dec. 29, 2015 is a system for logging biometric data. Singular biometric. It is a system used to scan and save a footprint image of an infant. Unlike the present invention, Roemen is not collecting all forms of biometric images, and is not collecting the biometric signature and medical data of men, women, and children for use in EMERGENCY situations to enable the immediate access of medical data to paramedics and medical authorities. Unlike the present invention, the system taught by Roemen is not configured for use in every-day life, such as going to a routine doctor's appointment. Additionally, Roemen does not teach the international implementation of the system. In contrast, the present invention is configured for use world-wide to facilitate the access of an individual's medical data from any location by medical personnel.

Similar substantial differences relate to U.S. Pat. No. 7,308,122, issued to McClurg on Dec. 11, 2007. McClurg has claimed a biometric imaging system and method. Unlike the present invention, McClurg scans fingerprints and not other forms of biometrics, and does not disclose a personal medical data system for emergency situations and everyday life to facilitate the retrieval of relevant medical data of individuals by physicians. Additionally, McClurg does not offer the additional safeguards of the present invention accompanied with a med+card (medical card) and a 911 application for all mobile devices.

U.S. Pat. No. 6,187,540, issued to Staub on Feb. 13, 2001, is for a method of newborn identification and tracking. Staub teaches the use of genotyping to maintain samples of the newborn and the mother. Unlike the present invention, Staub focuses on these cellular samples.

Currently, there is no system on the market that facilitates the rapid access of relevant medical data via a brief biometric scan of an individual to help doctors save time. Most importantly, there is no system that allows access to a contact number of family member or a friend/neighbor of the individual in real-time. Often, nurses and responders are forced to break into the individual's mobile phone or computer to look for a clue, wasting a lot of time and raising privacy concerns. There is no system on the market that allows access to the medical data as a universal platform (to all hospitals, doctors, paramedics, and other medical authorities) in order to promptly and properly make vital decisions in emergency situations.

As for right now, the method that we have available for identifying the patient in case of an emergency, is called DNR (Do Not Resuscitate) Medallion. Not many people even know that such a medallion exists. The DNR Medallion is not used by many people because it is not practical and many believe that the DNR Medallion process is antiquated and not efficient. There is a need for a solution to modernize the DNR Medallion.

Thus, there is a need for a system that facilitates the immediate access and retrieval of medical data of an individual—that enables all hospitals and medical personnel in real-time via a biometric scan, especially at the site of an emergency situation—to provide medical aid. Such a new system is preferably envisioned world-wide because if an individual travels in a foreign country for work or pleasure, and that individual is involved in an accident and is badly injured, all hospitals and doctors will have access to the universal medical emergency data system. In doing so, and having such a system as a universal platform internationally, it provides that each country guarantees the same rights, privileges, and aid in regard to emergency medical care. In other words, there is a need for a system that provides to all emergency responders, and all medical authorities, instant unfettered access to pertinent medical data for an individual (patient) in need via a biometric scan. This will rapidly identify an individual, regardless of the individual's location, and possibly help the doctors to make a vital decision at the scene of an emergency or during transportation to an acute care hospital. Such a system facilitates the identification of any incapacitated individual, even in the event of partial bodily decay. Such a system will not only help patients to communicate better and faster in real-time with physicians, dentists, pharmacists, radiologists, laboratories, hospitals, and health insurance companies, but will also help doctors and medical clinicians to keep a better and more accurate patient medical history that facilitates consistent updates to the medical records of the patient.

Additionally, such a system provides the option of an individual's choice to donate organs, and creates a paper-free system to facilitate such decisions. The ability to have the individual's advance instructions can be extremely useful under difficult circumstances. It can alleviate additional emotional suffering for the family and friends undergoing such a painful time—eliminating the burden for the family and friends of the patient to make such decisions under time pressure—in addition to the emotional suffering. There is a need to provide a contact during the scene of an emergency—rescue team, doctors, police officers, investigators often don't know who to contact at the scene of an emergency when the individual is in a critical condition or has just died.

As for now, we have medical employees and investigators often spending hours to look for a contact number of a family member, breaking into computers or mobile phones. There is a need for a system that solves that problem so that a contact number in the biometric medical data file of the individual is used in case of an emergency.

Also, there is a need to provide the instructions of individuals of any age, in other words of children too. For example, one patient Carlee from Texas, 2-time Heart Recipient, has a unique distinction. She is the first person to be fortunate to have received two heart transplants: the first time when she was 1½ years old and the second time when she was 13 years old. So young, coming close to death twice, an organ donor has given her—at a young age—a new meaning of life and giving back. Now at almost 16, she can't wait to get her driver license and sign up to register as organ donor. That raises a question: Why do we have her—or anyone else—wait to register as an organ donor when turning 16? We need to take into consideration that unfortunately accidents, injuries and death happen at any time and at any age, anywhere.

It is therefore advantageous to provide such a system for the transmission of an individual biometric identification linked with the individual pertinent medical data to be used during an Emergency when the individual is under critical condition and not able to communicate with first responders, paramedics, and all medical Authorities. There is a need for a system that develops an easier way to instantly access an individual's biometric identification and medical data needed during an emergency circumstance and when there is a need in everyday life.

In addition, there is a need for a system that provides, very importantly, the individual written specific instruction and unique wishes to efficiently identify in real-time, by Medical Authorities, at the time of a serious injury or death.

Recently, faces and hands have been added to the list of organs that can be successfully transplanted. In one specific case in 2001, Patrick Hardison had his face severely burned and completely disfigured during a search and rescue mission. Last year, he underwent the very first extensive face transplant, after a 26-year-old Brooklyn bike mechanic died after sustaining a brain trauma in a cycling accident. David's mother made the difficult decision to donate her son's organs—including his face—and changed Patrick's life forever. This November, Patrick got to thank David's mother in person for the first time in NY City during a very emotional meeting.

Transplantation is one of the great medical advances of our time. How does it work? It all starts when someone's organ begins to fail. And that person will need a transplant to survive. An evaluation is conducted at a transplant center. And if that person is a good candidate for a transplant, he or she will be put on the nation transplant waiting list. Once a person is on a list, the wait for the organ begins. A national system matches people on the waiting list with donors.

The factors considered in matching donors and recipients include: Blood Type, Body Size, How sick the patient is, Distance from Donor, Tissue Type, and length of Time on the waiting List.

There is no telling how long the wait will take. In fact, some people don't receive an organ in time. Because the waiting list is very long and there are not enough donors available, an average of 22 people die each day. Many of those are children. People don't think about that children get sick too.

There is a chance here to do something great. Imagine how many we can save if there were an invention that creates and organizes a system that lets the emergency first responders and medical authorities know in REAL-TIME of each individual's instructions (Family member contact during an emergency; Do Not Resuscitate instructions; Identify registered donors; Honor individual deceased instructions.)

Most of the transplants come from deceased donors. A person comes to a hospital with an injury from an accident, a stroke or lack of oxygen. The doctors work hard to save the people's lives, but sometimes nothing can be done. There is a complete irreversible loss of brain function. The patient is clinically and legally dead.

A person can give life—as many as 8 lives—through organ donation and improve the lives of 50 and many more, through eye and tissues donation. But now, minutes matter. Matches must be found, and transplants must happen quickly.

The hospital contacts an OPO, the OPO (Organ Procurement Organization) manages the organ recovery process. The OPO checks the State organ donor registry. If the person is already registered as a donor, the hospital will inform the family. If not, the hospital will ask the family to authorize donation. All that takes time. A medical examination takes place. The hospital checks the medical and social history of the individual. (Also, this process takes time) And if the person is eligible to be an organ donor, the computer begins to search on the national waiting list for well match patients. The best match patients for each organ are contacted by the transplant team. This is the call that every single person on that list is been hoping for. That call can mean a second chance of life. Now the transplant happens. A surgical team recovers the organs, the corneas and other tissues. The organs are sent to the transplant Hospital, where the patient and transplant team is waiting.

The goal here is to ideally eliminate steps that delay the process of the patient instructions and personal wishes being made known to facilitate the organ donor process.

More specifically, there is a need for a system that provides timely matter (in real-time) execution of the patient choice instructions and personal wishes during the scene of an emergency, which could be tremendously advantageous. As one can see, the traditional method requires a lot of steps and effort, and is time consuming. It is not working efficiently, when time is of the essence.

More specifically, after removal from a donor's body, a heart can survive just about four hours. Given the shortage of organ donors, most patients who are on the waiting list to receive a new heart, will not be able to get one; therefore, that patient will not make it when a heart transplant is the only option for a patient to survive.

Because medical technology has improved dramatically in the last couple of years, a liver can be stored on ice longer. However the shorter the ischemic or “on ice time”, the better for liver graft function. Therefore, depending on the organs, some factors become more important. Some organs can survive outside the body longer than others.

The demand of organs significantly surpasses the number of donors everywhere in the world. Because donor organs are in short supply, every day, there are more people waiting for a transplant than available organs.

If an individual wishes to donate their entire body, there are medical organizations of the individual's choice, and thus, there is a need for an invention with such a system, that provides arrangements in instructions. Medical schools, research facilities, and other agencies study bodies to understand how diseases affect human beings. This research is vital to saving and improving lives.

There are over 150,000 Americans waiting to receive an organ transplant. Millions of people die each year without signing up as organ or tissue donors. Every 10 minutes, an additional person appears on the transplant list. As such, an average of 22 people die each day because the organs they need are not donated in time. Statistics state that 90% of Americans support organ donation, but only 30% of them are known organ donors at the present time. There is a need for an invention to be instrumental to solve this problem.

Moreover, there is a need for such a system that can help to better combat fraud nationally, and is instrumental to preventing and alerting individuals to medical fraud. Fraud has often been a problem in the past. The complexity and occasional error in the medical field makes it even more challenging for people and special agents to keep track of what is going on regarding the treatment of an individual. For example, a doctor in Michigan misdiagnosed several patients with cancer, and made the national news. Many healthy patients became very sick, and some even died due to the treatment from the doctor, who prescribed them with expensive and unnecessary chemotherapy drugs. The government indicated that the doctor didn't simply lie to his patients, but he also manipulated them to keep them from finding out the truth of their condition. He took control of the patients, including controlling access to the patients' files by others, and remaining on call, even when other doctors were making rounds on his hospitalized patients. The doctor received a 175 year sentence, but unfortunately, the victims cannot be brought back. With the system of the present invention, America can prevent a repeat of this horrible type of torture and medical fraud.

If one takes into consideration that recently, the police were working on a case of an individual that was missing for 23 years. The individual, Hoagland, as it turned out, was found in Florida after having stolen a death certificate from a fisherman who had died. Hoagland used the death certificate to get a birth certificate and drivers' license in the name of the deceased man, and built a different identity. And in this particular case, a nephew who was researching his family's history on the internet, discovered a new pilot's license assigned to his “dead” uncle—the deceased whose identity had been stolen by Hoagland.

This would have never happened if there was a universal biometric system in place as described as needed above, as there it would have been discovered immediately that such an individual, Hoagland, was not in fact, Terry Symansky (the deceased), since their biometric fingerprints would never have never matched!

As one can see, another factor to consider is that there is a need for a system that can be extremely advantageous for many different reasons. This could tremendously help against terrorists possibly stealing someone else's identity to commit other terrorist attacks.

### SUMMARY OF THE PRESENT INVENTION

The present invention is a global biometric-authenticated medical data and emergency access system for use by authorized paramedics, first responders, doctors, all hospitals, medical professionals, and the general public—whoever may choose to be part of this system—as a universal platform in the U.S. and internationally. The system employs a biometric authentication and identification mechanism which links an individual's biometric scan to his or her medical data, medical history, medical records, and emergency contact information. The medical data of the individual may include the individual's name, age, blood type, allergies, medication (the kind), and dental records—in other words, the most valuable medical information associated with the individual's most recent medical record. For example, the individual's medical condition, such as diabetes, asthma, or a particular blood pressure—or even the most recent surgery that the individual is known to have undergone, is greatly useful for the prompt emergency aid procedures and for successful diagnosis assistance.

A mobile application component of the present invention allows, if faster, an easy way to call for help under difficult circumstances. For example, if the individual is involved in an accident, and has difficulties communicating, the mobile application will be handy. Further the mobile application exists to permit the individual with access to his or her own records, and facilitates the alteration of emergency contact information. A 911 component of the mobile application enables the individual, in case of an emergency, to easily be connected with 911 services, and for the location of the individual to be transmitted to the authorities. The location is derived from the onboard GPS of the mobile device of the individual, and/or service triangulation.

A fingerprint sensor may be particularly advantageous for identity verification and/or authentication in an electronic device, and more particularly, a portable device. Such a fingerprint sensor may be carried by the housing of a portable electronic device and may be sized to sense a fingerprint from a single-finger or multiple fingers. Biometric technology works by comparing a specific portion of the human body with the data on a file for purposes of authentication, identification, and in this particular case, medical uses. Biometric scanning devices are now available for use wirelessly, providing for mobile or stationery use by governments, the FBI, and law enforcement agencies around the world. Finally, thanks to advanced and sophisticated technologies including advanced secure database design and encryption, the present invention will enable all hospitals, doctors and all medical personnel, including paramedics, to use this system to rapidly attain pertinent medical data to better assist any individual. It should be noted that the biometric devices of the present invention do not actually store fingerprint images. Instead, they save a mathematical representation of the individual's biometric data (signature). When a biometric device scans a fingerprint or a hand during a supervised enrollment process, only an encrypted mathematical representation of the fingerprint is stored. The algorithm cannot be reconverted to an image, so no one can duplicate the fingerprint. As a result, it is virtually impossible to duplicate the original image. This is unlike a passport or identification card that could easily be stolen.

A goal of the present invention is to create a universal, new platform for the entire medical emergency system in the U.S., as well as abroad. Health records are some of the most valuable personal documents. A confusion of papers can be attributed to a missed diagnosis, which can leave room for error. It is difficult to imagine a future in which medical records are not accessible via cloud storage such as a central clouding government database. As such, the system of the present invention is envisioned to be a part of a government database, employing security safeguards of the data housed within cloud storage. Doctors around the world need access to them quickly, and they need to be accurate in order to save lives, and provide expedient treatments. A lapse in security and improper accounting for changes can mean the difference between life and death for a patient. It is envisioned that, in order for the system of the present invention to successfully work, the entire U.S. would need to adopt it, and ideally, the rest of the world may follow. As such, the present invention is envisioned as a universal platform.

One important use of the system of the present invention is for the timely identification of victims of catastrophe or tragedy. For instance, with the most recent tsunami and violent tornados, there has been a problem identifying the deceased. Bodies are often found without any means of identification on them, and their biometric data is not always logged. The system of the present invention solves this issue by providing an efficient and expedient means by which an individual, living or deceased, may be identified.

For example, in the event that an individual is involved in an accident or a tragedy of any kind, the following scenarios can occur:

1. The individual is unconscious, and/or bleeding heavily. The individual is still alive. When there are sirens and IV's, chest compressions, there is no time to look for clues. Every second counts when saving lives.

2. The individual is almost dead.

For example, in the event that an individual is very badly injured and he or she is unconscious, without a pulse, and not breathing at this point, does the individual wish to be resuscitated? If the individual does not wish for resuscitation, and opts for no artificial nutrition, and no feeding tubes, the desires of the individual should be honored. If the individual does not wish to suffer and become a burden to anyone, wishing to die with dignity, then the system of the present invention ensures that the wishes of the individual are honored.

3. The individual is deceased.

In such an instance, if the paramedics find the individual deceased, the system of the present invention provides a means of relaying the individual's instructions upon death. Such instructions could include, “I give you permission to use my organs, tissues, and/or parts of my body to who most need it and for those that are currently in a critical condition list.” Or for example: “I wish to donate my body after I die to a Research Medical Institute. (Name of the Institute).” There are millions of people in critical conditions. A deceased individual could save up to eight people when the remains of the individual are donated for organ transplant. A novelty of the present invention is the increase of organ donors in the nation, and possibly worldwide.

Additionally, another important use of the system of the present invention is that it can be used to help find a missing person such as a child. If a child gets lost or is kidnapped, it may be likely that there is no record of the child's fingerprints that are rapidly accessible.

The present invention creates an optimal system in regards of security, safety and privacy. Conversely, a password can be lost or forgotten, however a fingerprint cannot. The use of biometric authentication is also instrumental in preventing identity theft. Such a system would be instrumental in solving issues including DNR and after-death instructions, which are currently often a problem. It can be quite overwhelming to be asked to make health care decisions for another individual who is dying. Therefore, the system of the present invention can help to solve this need.

### BRIEF DESCRIPTION OF THE DRAWINGS

The present invention will be better understood with reference to the appended drawing sheets, wherein:

FIG. 1 displays a flow chart depicting the process of use of the system of the present invention.

FIG. 2 exhibits a top view of a conventional biometric device of the present invention.

FIG. 3 shows a front view and a rear view of the medical card of the present invention.

FIG. 4 depicts a view of the 911 emergency button of the present invention installed in the control center of a mobile device by a mobile device manufacturer.

FIG. 5 displays a screenshot of the mobile device application component of the present invention, showing the data display and edit screen.

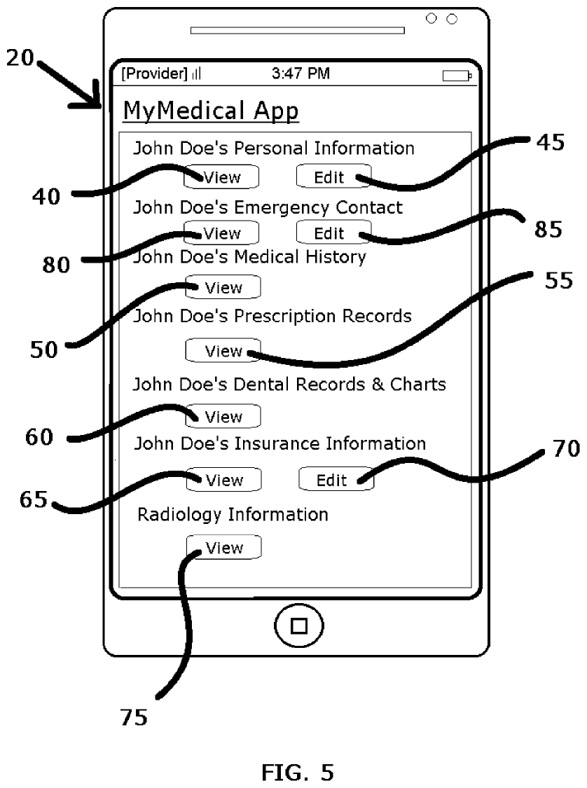

FIG. 6 exhibits a 911 app downloaded by the user on his or her smartphone.

### DETAILED DESCRIPTION OF THE PREFERRED EMBODIMENT

The present invention involves the creation of a mechanism to be used during a critical emergency—and in general when needed—wherein an individual's biometric identification links to his or her medical data, and is stored into a digital coded data application via safe guards and database clouding. By scanning and saving an individual's fingerprint, linked to his or her personal medical information, and combining these items into a compartmentalized file, the present invention ensures that the fingerprint image is accurately matched with the correct medical data of the individual. The data and images collected are either saved in the memory storage or transmitted wirelessly to a computing device or hard drive. Once all of the images and data are collected, saved and scanned by the portable biometric device, a file can be transferred to a dedicated record storage system/cloud. The file is initially saved or stored in the memory storage of the portable biometric device, and can be transferred to the record system via conventional means. This includes a USB, SD/MD card, or other like transfer via a conventional wireless transmitter operating in communication with a router system connected to the record system.

The present invention is a computerized Emergency medical data reference system equipped with multiple components. Data of the system is identity-restricted, and requires physical biometric authentication at the point-of-use for access. The system of the present invention provides the mechanism for the biometric signature of an individual to be intrinsically linked to (and facilitating the access thereof) the medical data and identity data of that individual, including but not limited to: the individual's name, age, blood type, allergies, medical history, medical records, family hereditary record history, dental records, dental charts, pharmaceutical records, lab results, radiology information, prescription information, prescription history, OTC medications, health insurance information, DNA (if sequenced), and emergency contact information. Additionally, the medical data includes a DNR (Do Not Resuscitate) clause of the individual, if applicable, as well as after-death instructions. The lack of donors in America is currently a large problem. This is mostly due to the fact that people do not think about death often, and when death happens, it is too late for action. In addition, it is anticipated that the system of the present invention is to be instrumental for the increase of organ donors in the U.S., as well as abroad. The lack of donors in America is currently a large problem. The system of the present invention is envisioned to be an extraordinarily advantageous means to solve this issue.

Advantages of the present invention should be understood to be the following:
Keep an updated and accurate medical record data of all individuals to avoid possible errors and wrong diagnoses, especially in emergency situations. Allow access for a prompt responder when emergency medical assistance is required.
Save precious time for doctors and to help them to make vital decisions in an emergency.
Combat fraud nationally
Identify unknown victim
Help to find missing individuals and children via biometric identification.
DNR Instructions.
After-death Instructions.
Increase organs donors in the nation and worldwide.
Identify “in real-time” registered organ donors.
Honor individual deceased instructions.

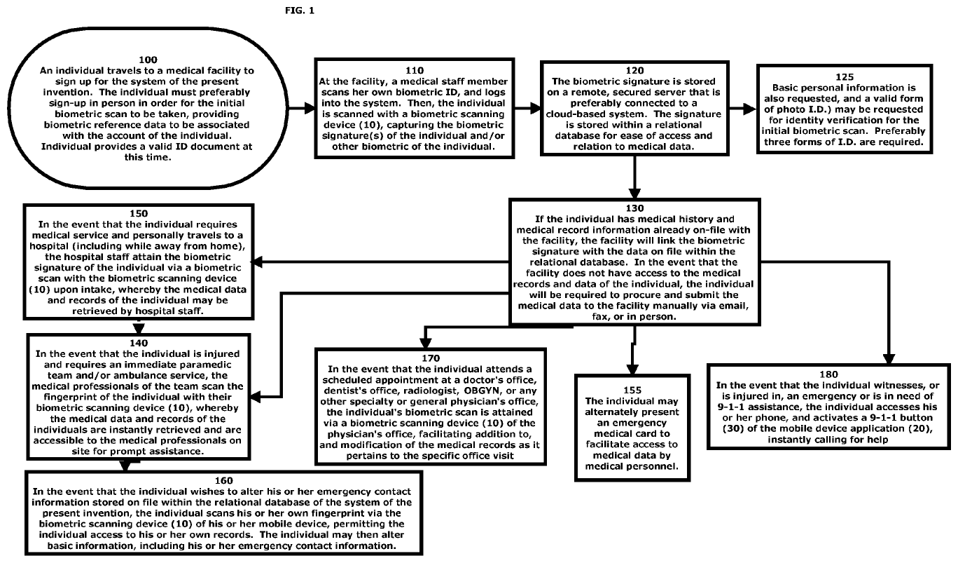

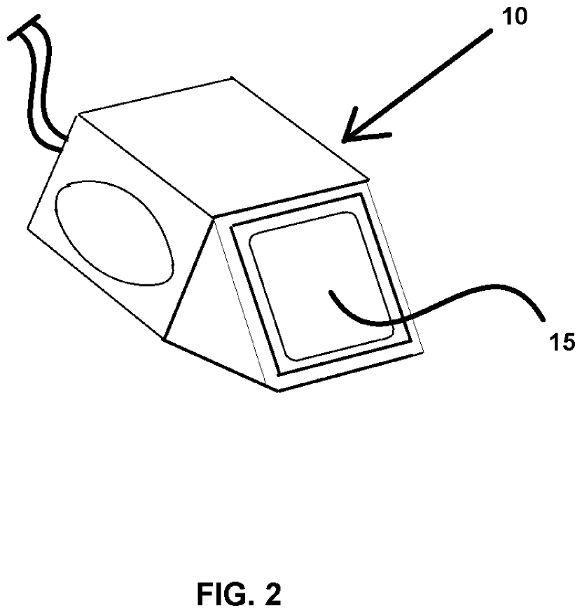

A first component of the present invention is a biometric scanning device (10) configured to quickly and easily scan/read, capture, and record biometric data from an individual. Embodiments of the biometric scanning device (10) employed by the system of the present invention can be seen in FIG. 1 and FIG. 2. The biometric scanning device (10) is preferably a conventional reader and scanner device. The biometric scanning device (10) is preferably a portable, internet-connected fingerprint scanner, which may be embedded in a larger mobile device. Alternate embodiments of the present invention may employ a deep-vein scanner as an alternate means of capturing and recording unique biometric data. Similarly, other alternate embodiments of the present invention may employ an iris or retina scanner as a means of biometric authentication. It is envisioned that other forms of biometric authentication may be used in the future that are cost-prohibitive at present, including facial recognition, iris recognition, palm vein recognition, or others.

A conventional fingerprint reader is fairly simple to use, even when time is limited. The individual places his or her finger on a window (15) of the biometric scanning device (10), and the reader automatically scans the fingerprint of the individual. A light and/or audio conventionally indicates that a fingerprint image has been captured successfully. On board electronics calibrate the reader, and encrypt the scanner data before sending it over the USB or wireless interface. The biometric reader and the fingerprint recognition engine have an extraordinary ability to authenticate even the most difficult fingerprints accurately and rapidly, acquiring any individual's fingerprint in virtually any condition. Fingerprint scanners are considered among the more secure methods of data access, as they do not rely on items that can be forgotten or lost, such as tokens, keycards, or text-based passwords.

In the preferred embodiment of the present invention, the fingerprint of the individual is not stored locally on the biometric scanning device (10) or the computing device powering and processing the signal/scan from the biometric scanning device (10). Instead, the system of the present invention employs an algorithm that uses a unique set of data points found within the fingerprint of the user to uniquely identify each individual, and the data points are then stored, preferably, in a government cloud. Single or multiple fingerprints can be enrolled into the system depending on the preference of the individual. The set of data points are unique for each fingerprint. In this manner, the unneeded dissemination of the fingerprint of the user is prevented.

Once all of the medical data is stored in the file of the patient, linked to the individual's biometric signature, the data will remain in the system such that it is easy to access at any time for all medical authorities when there is a need. A biometric match via the biometric scanning device (10) will allow authorized medical professionals access to the patient's medical data file to all doctors. The authorized medical professional will login their medical employee ID patch and/or his or her biometric identification into the system before assisting any individual. A non-match of the biometric data will not provide access to the medical data file. It is envisioned that the individual will initially sign up for use of the system of the present invention physically, in-person, either at a government agency, doctor's office, or other private company such as a medical authorized facility, so as to capture the biometric signature of the individual on-site in a setting that can be conducive to the authentication of the identity of the individual, and register the individual's biometric signature (fingerprint) with the individual's medical data. It is envisioned that a small fee may be assessed for such a service, similar to when an individual travels to the DMV to obtain a government ID card or Driver's License card, and registers his or her fingerprint biometric there as well. Before the initial scan of the individual signing up for the system of the present invention at intake, it is envisioned that the medical personnel must first log-in with his or her medical employee ID card/patch, and/or with his or her biometric signature in order to assign an identity to the individual processing the intake.

The process of initial set-up and subsequent use of the system of the present invention, as depicted in FIG. 1, is preferably as follows:
1. An individual travels to a medical facility to sign up for the system of the present invention with two forms of valid ID and/or birth certificate. The individual must preferably sign-up in person in order for the initial biometric scan to be taken, providing authentic biometric reference data to be associated with the account of the individual. (100) The individual provides a valid ID document at this time and/or birth certificate.
2. At the facility, a medical staff member scans his or her own biometric ID, and logs into the system before assisting individuals in the process. Then, the individual is scanned with a biometric scanning device (10), capturing the biometric signature of the individual. (110) The biometric signature may be captured as a fingerprint (1-10 at the individual's option), deep vein reading, iris or retina scan, brain scan, or other unique biometrics.
3. The biometric signature is stored on a remote, secured server that is preferably connected to a cloud-based system (such as those managed by government agencies). The signature is stored within a relational database in relation to medical data. (120) It is envisioned that the biometric signature acts as a private key for access to the medical data of the relational database. Basic personal information is also requested for the individual to register for the system, and valid forms of photo I.D. (such as a birth certificate, driver's license, or passport) may be requested for identity verification for the initial biometric scan. Preferably three forms of I.D. are required. (125)
4. If the individual has medical history and medical record information already on-file with the facility or doctor's office, the facility will link the biometric signature with the data on file within the relational database. In the event that the facility does not have access to the medical records and data of the individual, the individual will be required to procure and submit the medical data to the facility manually via email, fax, or in person. (130) In other cases, the facility can collect the individual's blood to determine the blood type and other medical data upon enrollment of the individual. This is helpful in the event that the individual has just moved from another country or region, and does not presently have all of his or her medical data available.
   The system of the present invention may now be employed in a variety of scenarios including:
5. In the event that the individual is injured and requires an immediate paramedic team and/or ambulance service, the medical professionals of the team (after logging into the system with their medical employee ID and/or biometric identification) scan the fingerprint of the individual with their biometric scanning device (10), whereby the medical data and records of the individuals are instantly retrieved and are accessible to the medical professionals on site for prompt assistance. (140) This remains true in scenarios in which the individual is conscious or unconscious. Because of the advantage of the instant access of the medical data of the individual, the paramedics can communicate instantly with the hospital's doctor in real-time. For example, if the individual has a rare blood type, the doctor at the hospital can immediately place a request to get that particular rare blood as soon as possible, saving precious time.
6. In the event that the individual requires medical service and personally travels to a hospital (including while away from home), the hospital staff attain the biometric signature of the individual via a biometric scan with the biometric scanning device (10) upon intake, whereby the medical data and records of the individual may be retrieved by hospital staff/personnel. (150) The individual may alternately present the emergency medical card to facilitate access to medical data by medical personnel. (155)
7. In the event that the individual wishes to alter his or her emergency contact information stored on file within the relational database of the system of the present invention, the individual scans his or her own fingerprint via the biometric scanning device (10) of his or her mobile device, permitting the individual access to his or her own records. The individual may then alter basic information but not medical records and such, including his or her emergency contact information. (160) This information is relevant and available to all parties, including all doctors, dentists, medical professionals, etc.
8. In the event that the individual attends a scheduled appointment at a doctor's office, dentist's office, radiologist, OBGYN, or any other specialty or general physician's office, the individual's biometric scan is attained via a biometric scanning device (10) of the physician's office, facilitating the addition to, and modification of, the medical records as it pertains to the specific office visit. (170)
9. In the event that the individual witnesses, or is injured in, an emergency or is in need of 911 assistance, the individual accesses his or her phone, and activates a 911 emergency button (30) of the mobile device application (20), instantly calling for help. (180)

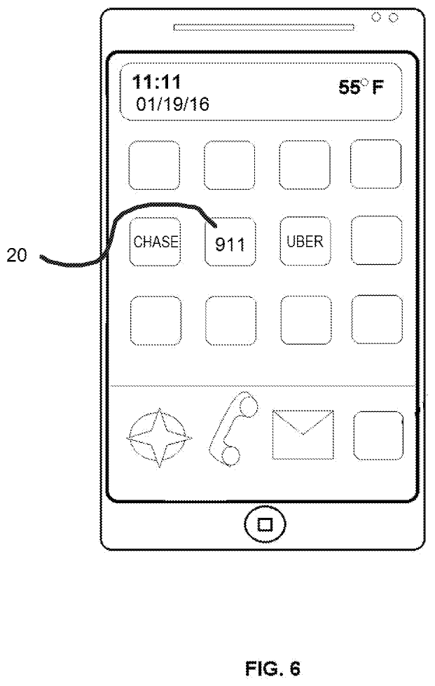

A second component of the present invention is the mobile application (20) as shown in FIG. 6. The mobile application (20) of the present invention, available to be downloaded by the user, is preferably locked via the biometric signature of the individual (owner of the data). As such, it is envisioned that the mobile application (20) of the present invention is configured to operate best on smartphones and tablet devices equipped with a fingerprint reader as a biometric scanning device (10). However, other embodiments of the mobile application (20) of the present invention may be configured to function without an onboard biometric scanning device (10).

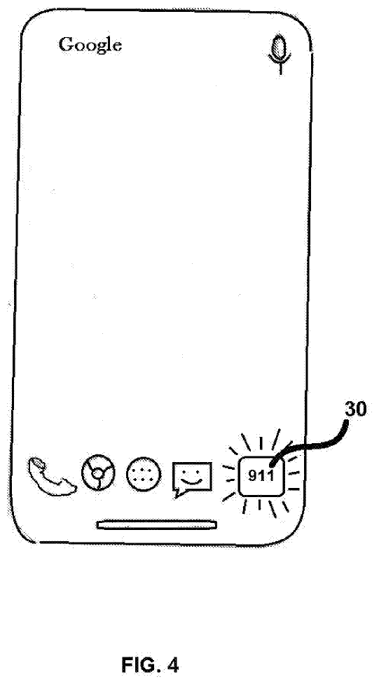

There is also a 911 emergency button (30) of the present invention, as shown in FIG. 4. It is envisioned that the 911 emergency button (30), when activated, instantly contacts 911 services locally, and transmits the user's location as obtained from the on-board GPS or cellular triangulation of the mobile device. By pressing and holding the 911 emergency button (30), the user will be instantly linked as a shortcut to an emergency dispatch center, which can send emergency responders to the user's location in an emergency. The 911 emergency button (30) may require a ‘press-and-hold’ activation mechanic on the bar screen control center on most mobile devices (phones) equipped with a touch screen per convention, in order to prevent accidental activation. The manufacturer can dispose the 911 emergency button (30) on the lock screen in addition to the home screen, or the settings dropdown or slide menu of the mobile device.

Note that the 911 emergency button (30) is shown as ‘911’ as an icon in FIG. 4. However, in the preferred embodiment of the present invention, the 911 emergency button (30) is preferably installed by the mobile device manufacturer during initial set-up of the device, or in other words, hard-coded into the control center of the mobile device. It is envisioned that the manufacturers of mobile devices may provide the installation of the 911 emergency button (30) on the lock screen of all mobile devices for customers to use as a global feature of the OS of the mobile device. The user may download the mobile application (20) to his or her home screen that will allow the user to view and edit the user's profile and information. Because the 911 emergency button (30) needs to be easily accessible at any time, the phone manufacturers may opt to add the 911 emergency button (30) to the control center of the mobile device for a faster way for users to locate the icon for the 911 emergency button (30), which simply requires a quick vertical swipe for most Apple™ or Android™ devices. This particular feature of mobile devices is preferably colored red and/or white in color on the screen, and/or equipped with a special light on the screen of the device, to help the 911 emergency button (30) to stand out.

Additionally, the 911 emergency button (30) is preferably accessible by the user even when the phone is locked, and is preferably disposed at the top or the bottom of the screen of the mobile device. Because the 911 emergency button (30) preferably needs to be easily and rapidly accessible in case of an emergency, the manufacturers may consider installing this as a new feature in all manufactured mobile devices on the bar screen control center for customers to use as a global feature. It is preferably envisioned that device manufacturers dispose the 911 emergency button (30) in all phone devices on the lock bar screen as an easy way for the users to rapidly call for immediate help under difficult circumstances. It is envisioned that even if the mobile device is locked by the user (with his or her private security code or fingerprint), the 911 emergency button (30), is still preferably accessible to the user simply via a swipe. For example, in most Apple™ iPhone™ or Android™ devices, the 911 emergency button (30) merely requires a quick vertical swipe on the touch screen, and then a press-and-hold on the screen to be instantly connected to an emergency dispatch center. It should be understood that the 911 emergency button (30) of the present invention is envisioned to be available or installed to all mobile devices, not only on smartphones, and may be incorporated in tablets such as iPad™ devices. These incorporations or installations may be hard-coded to the OS of such devices.

From within the mobile application (20) of the present invention, the user (patient/owner of medical data, and preferably, the owner of the mobile device) is able to view current medical data accessible to the system of the present invention, as well as edit certain components including the personal information of the user, emergency contact data of the user, and insurance information of the user. As such, the mobile application (20) is preferably equipped with a personal information view button (40), a personal information edit button (45), an emergency contact view button (80), an emergency contact edit button (85), a medical history view button (50), a prescription record view button (55), a dental records and charts view button (60), an insurance information view button (65), an insurance information edit button (70), and a radiology information view button (75). Each view button allows the user to view (and not edit) his or her medical data. It is envisioned that only the personal information, emergency contact information, and insurance information may be edited by the user. All other data pertaining to medical records may only be modified by authorized medical personnel with appropriate clearance.

Alternate embodiments of the present invention include variations on type of biometric scanning device (10) employed by the system, variations on the type of relational database employed, and variations on the size and scope of the system regionally. It is envisioned that the preferred embodiment of the present invention is a global system, wherein an individual may be injured and incapacitated in any country, and the medical personnel of the local region may quickly access the medical data of the individual from his or her home country/region by attaining the biometric signature of the individual via the biometric scanning device (10).

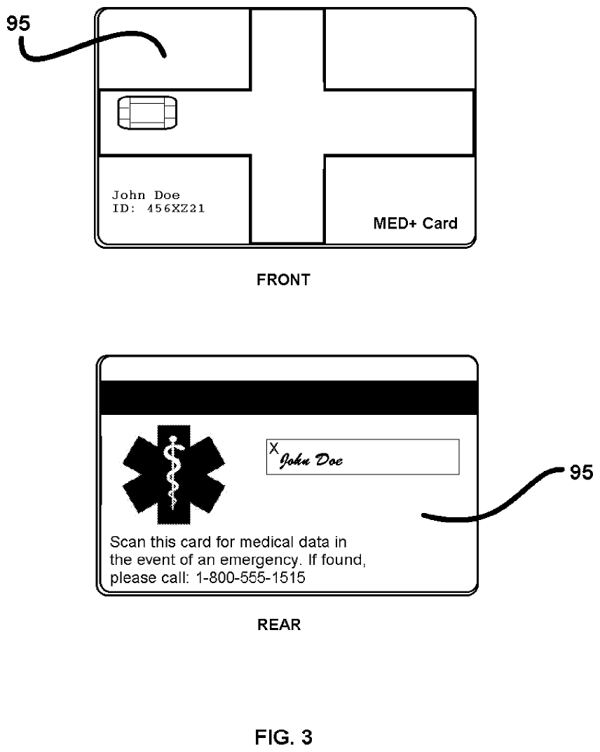

In addition, the present invention offers a physical medical card (95), which serves as a repository for the medical data made accessible via the present invention. As such, the medical card (95) of the present invention is configured to contain basic, primary medical data of the patient that is generally perceived as static, such as allergies, emergency contact data, blood type, medications (required, prescribed, or over-the-counter), personal information, and other similar data—but not the biometric medical emergency medical data system described above. The medical card (95) of the present invention may be equipped with a microchip for enhanced security. In some embodiments of the present invention, the data of the medical card (95) is configured to be updated each time the medical card (95) is scanned. The medical card (95) may be useful in areas of the world lacking a decent internet connection, infrastructure, or power systems. In such scenarios, medical personnel can employ the medical card (95) to provide basic medical history and data of the individual in an emergency. The medical card (95) of the present invention is preferably red in color with a white cross, as shown in FIG. 3. The name of the owner of the card may appear the medical card (95), and/or a card ID number could be present on the front of the card. A magnetic strip, as well as other information is preferably disposed on the rear of the card, as shown in FIG. 3. A microchip is also preferably disposed in the medical card (95). A biometric fingerprint is also, preferably, envisioned to appear on the medical card (95). It is to be understood that the physical medical card (95) is not in place of the biometric discussion of the present invention as aforementioned, but is actually an enhancement to the biometric discussion of the present invention to be used in cases where internet connections are not readily available.

Additionally, users are preferably provided the option to include any medical-related preferences within their medical data profile, such as information that does not directly relate to medical data/records. For example, preferences can be included that indicate preferred emergency contact names and numbers to be used according to certain times of year, or times of day. Additional preferences may also be included pertaining to preferred locations of treatment, DNR data, and other user-controlled preferences not dictated by medical data or records. These preferences may include specific directives to be followed by medical personnel when the need arises.

Additionally, it should be understood that the medical card (90) of the present invention may be configured to automatically update/synchronize with the app of the present invention in order to maintain the most up-to-date data. Similarly, the medical card (90) may be synchronized manually on-demand when requested.

It should be understood that the 911 emergency button (30) of the present invention is configured to automatically dial 911, or the appropriate regional emergency number where applicable.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A method for the dissemination of emergency specific medical data of an individual to be used during an emergency situation at an emergency location outside of a medical facility via a biometric scan, the method comprising:
receiving, at a secured network server, registration information from a biometric scanning device via a network, the registration information capturing an association between the individual and first biometric scan data of the individual and including an initial biometric authorization scan of a person inputting the registration information, the first biometric scan data including a first numerical algorithm conversion of the biometric scan of the individual;
associating, at the secured network server, the registration information with a user profile;
setting, at the secured network server, the registration information as a biometric baseline signature unique to the individual;
storing, within a secured relational database at the secured network server, the biometric baseline signature;
searching the secured relational database for emergency specific medical data associated with the first numerical algorithm;
linking, within the secured relational database, the emergency specific medical data associated with the first numerical algorithm within the user profile;
receiving, at the secured network server, second biometric scan data from a biometric scanning device during the emergency situation, the second biometric scan data including a second numerical algorithm conversion of biometric scan of the individual during the emergency situation;
comparing, at the secured network server, the first numerical algorithm conversion and the second numerical algorithm conversion;
outputting, to a device at the emergency location, in response to a match between the first numerical algorithm conversion and the second numerical algorithm conversion
the emergency specific medical data within the user profile, and
one or more directives associated with the output emergency specific medical data, the one or more directives including do-not-resuscitate information, allergy information, after-death instructions, organ donation information, and family emergency contact information;
relaying at least some of the output emergency specific medical data and the one or more directives from the emergency location to the medical facility prior to arrival of the individual at the medical facility; and
updating the emergency specific medical data of the individual based on the medical care services performed during the emergency situation.

2. The method of claim 1, further comprising:
saving the updated emergency specific medical data to the database of the secured server.

3. The method of claim 1, wherein the secured server is a cloud-based server.

4. A system for the dissemination of emergency specific medical data of an individual to be used during an emergency situation at an emergency location outside of a medical facility via a biometric scan, the system comprising:
a first biometric scanning device; and
a secured network server configured to
receive registration information from the first biometric scanning device via a network, the registration information capturing an association between the individual and first biometric scan data of the individual and including an initial biometric authorization scan of a person inputting the registration information, the first biometric scan data including a first numerical algorithm conversion of the biometric scan of the individual,
associate the registration information with a user profile,
set the registration information as a biometric baseline signature unique to the individual,
store, within a secured relational database at the secured network server, the biometric baseline signature,
search the secured relational database for emergency specific medical data associated with the first numerical algorithm,
link, within the secured relational database, the emergency specific medical data associated with the first numerical algorithm within the user profile,
receive second biometric scan data from a second biometric scanning device during the emergency situation, the second biometric scan data including a second numerical algorithm conversion of biometric scan of the individual during the emergency situation,
compare the first numerical algorithm conversion and the second numerical algorithm conversion,
output to a device at the emergency location, in response to a match between the first numerical algorithm conversion and the second numerical algorithm conversion
the emergency specific medical data within the user profile, and
one or more directives associated with the output emergency specific medical data, the one or more directives including do-not-resuscitate information, allergy information, after-death instructions, organ donation information, and family emergency contact information, at least some of the output emergency specific medical data and the one or more directives being relayed from the emergency location to the medical facility prior to arrival of the individual at the medical facility, and
update the emergency specific medical data of the individual based on the medical care services performed during the emergency situation.

5. The method of claim 1, wherein updating the emergency specific medical data includes updating the secured network server, the secured network server being at a location different from the emergency location.

6. The method of claim 1, wherein the outputting includes outputting, to a device at the emergency location, in response to a match between the first numerical algorithm conversion and the second numerical algorithm conversion:
the emergency specific medical data within the user profile, and
one more directives including do-not-resuscitate information, allergy information, after-death instructions, organ donation information, and family emergency contact information.
